(19) United States
(12) Patent Application Publication
(10) Pub. No.: US 2020/0188583 A1
(43) Pub. Date: Jun. 18, 2020

(54) RINGER'S SOLUTION MONITORING APPARATUS

(71) Applicant: Sinsung TK Co., Ltd., Gwangju (KR)

(72) Inventor: Eun Suab LIM, Gwangju (KR)

(73) Assignee: Sinsung TK Co., Ltd., Gwangju (KR)

(21) Appl. No.: 16/574,934

(22) Filed: Sep. 18, 2019

(30) Foreign Application Priority Data
Dec. 13, 2018 (KR) 10-2018-0161108

(51) Int. Cl. A61M 5/168; A61M 5/14

(52) U.S. Cl. CPC A61M 5/1689; A61M 5/1417; A61M 2205/3313; A61M 2205/3389; A61M 2205/3584; A61M 2205/8206; A61M 2205/8237; A61M 2205/0216

(57) ABSTRACT

The present invention relates to a Ringer's solution monitoring apparatus in which a dropper is installed and which detects Ringer's solution input information such as an input speed and an input amount of a Ringer's solution and informs a manager of the Ringer's solution input information. The Ringer's solution monitoring apparatus includes a main body, a dropper attachment part which is provided on the main body and to which a dropper is attached, a Ringer's solution detector which detects a Ringer's solution falling in the dropper attached to the dropper attachment part, and a fixing unit which detachably fixes the main body to a hanger on which a Ringer's solution container is hung.

### CROSS-REFERENCE TO RELATED APPLICATION

This application claims priority to and the benefit of Korean Patent Application No. 10-2018-0161108, filed on Dec. 13, 2018, the disclosure of which is incorporated herein by reference in its entirety.

### BACKGROUND

### 1. Field of the Invention

The present invention relates to a Ringer's solution monitoring apparatus, and more specifically, to a Ringer's solution monitoring apparatus in which a dropper is installed and which detects Ringer's solution input information such as an input speed and an input amount of a Ringer's solution and informs a manager of the Ringer's solution input information.

### 2. Discussion of Related Art

Ringer's solutions accommodated in Ringer's solution containers (Ringer packs and Ringer bottles) are injected into blood vessels of patients so as to supplement the patients with nutrients (that is, glucose), injectable solutions, and the like for treatment and management for the patients. Generally, the Ringer's solutions in the Ringer's solution containers are supplied to the patients through supply tubes inserted into the patient's blood vessels, and the droppers are installed between the Ringer's solution containers and the supply tubes.

When such a Ringer's solution injection apparatus is used, there is inconvenience in that nurses or caregivers should always stay around the patients to frequently check an amount of Ringer's solution to block the Ringer's solution from being input and to replace the Ringer's solution container. In addition, in a case in which a time at which the Ringer's solution is removed and replaced has passed by mistake, since air passes through the tube which injects the Ringer's solution, and is injected into the blood vessel through the tube, a dangerous situation can occur, and thus a detection apparatus configured to monitor injection of the Ringer's solution and to inform of an injection end time is needed.

In order to solve such a problem, various Ringer's solution monitoring apparatuses have been developed as disclosed in Korean Patent Registration No. 10-0802313. However, in the case of the conventional Ringer's solution monitoring apparatus, since a separate holder such as a Ringer's solution container holder is required to detect a Ringer's solution discharged from a Ringer's solution container, it is cumbersome, and there is a disadvantage in that the holder disturbs activity of a patient.

In addition, when the monitoring apparatus is fixed to a hanger to install the monitoring apparatus, since a fixing method is inconvenient, there is a problem in that detachment and attachment of the monitoring apparatus is not easy, and since a standard of the Ringer's solution hanger is not fixed, there is a problem in that the monitoring apparatus may not have a structure compatible with hangers having different diameters.

### SUMMARY

The present invention is directed to providing a Ringer's solution monitoring apparatus to which a dropper is coupled and which is capable of measuring an injection amount and an injection speed of a Ringer's solution using Ringer's solution drops passing through the dropper.

The present invention is also directed to providing a Ringer's solution monitoring apparatus which is capable of being compatible with various Ringer's solution hangers which have different diameters and which are easy to detach and attach.

According to an aspect of the present invention, there is provided a Ringer's solution monitoring apparatus including a main body, a dropper attachment part which is provided on the main body and to which a dropper is attached, a Ringer's solution detector configured to detect a Ringer's solution falling in the dropper attached to the dropper attachment part, and a fixing unit configured to detachably fix the main body to a hanger on which a Ringer's solution container is hung.

The main body may have a predetermined inner space therein, and the Ringer's solution detector may be attached into the main body.

The dropper attachment part may include an inlet groove formed to be recessed a predetermined depth in an inward direction such that the dropper is inserted into one side of an external surface of the main body, the Ringer's solution detector may include an optical sensor configured to determine whether the Ringer's solution is falling in the dropper using light, and a light emitter and a light receiver may be formed in a surface of the inlet groove to emit light toward the dropper which has entered the inlet groove and to receive light.

The dropper attachment part may further include a dropper pressing part configured to press a surface of the dropper which has entered the inlet groove and to maintain a state in which the dropper has entered the inlet groove, and the dropper pressing part may include an inlet opening formed in a direction corresponding to an opening of the inlet groove such that the dropper enters and may be formed such that both ends of the dropper pressing part are opened outwardly along an external diameter of the dropper and are elastically deformable.

The dropper pressing part may include a plurality of dropper pressing parts formed to be vertically spaced apart from each other in the inlet groove.

The fixing unit may include a fixing support which is formed at one side of a rear surface of the main body and wraps around one side of an outer circumferential surface of the hanger, a rotating support which is formed to be rotatable on the rear surface of the main body and wraps the outer circumferential surface of the hanger with the fixing support, and an elastic member which elastically biases the rotating support in a direction in which the hanger is pressed such that a state in which the main body is fixed to the hanger is maintained by maintaining a state in which the fixing support and the rotating support press the outer circumferential surface of the hanger.

The fixing support and the rotating support may be allowed to wrap around the hanger regardless of an external diameter of the hanger because the rotating support rotates, and a friction pad may be formed on a contact portion in contact with the outer circumferential surface of the hanger so as to increase a frictional force.

The Ringer's solution monitoring apparatus may further include a communication module which transmits detected information detected by the Ringer's solution detector, and a managing module which is installed in a wireless terminal of a user and receives and displays the detected information transmitted from the communication module through the wireless terminal of the user.

Any one wireless communication method among Bluetooth, Bluetooth beacon, and Wi-Fi is applied to the communication module.

A battery configured to supply power for driving the Ringer's solution detector may be installed in the main body, a charging terminal may be provided on a side surface of the main body to charge the battery, and a cradle in which a coupling terminal in contact with the charging terminal of the main body is provided and into which the main body is inserted to charge the battery may be further included.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a view illustrating a state in which a Ringer's solution monitoring apparatus according to the present invention is used;

FIG. 2 is a perspective view illustrating the Ringer's solution monitoring apparatus of FIG. 1;

FIG. 3 is an exploded perspective view illustrating the divided Ringer's solution monitoring apparatus of FIG. 2;

FIG. 4 is a block diagram illustrating a configuration of the Ringer's solution monitoring apparatus of FIG. 2;

FIG. 5 is a perspective view illustrating a dropper fixing part of the Ringer's solution monitoring apparatus of FIG. 2; and

FIGS. 6 and 7 are perspectives view illustrating a fixing unit.

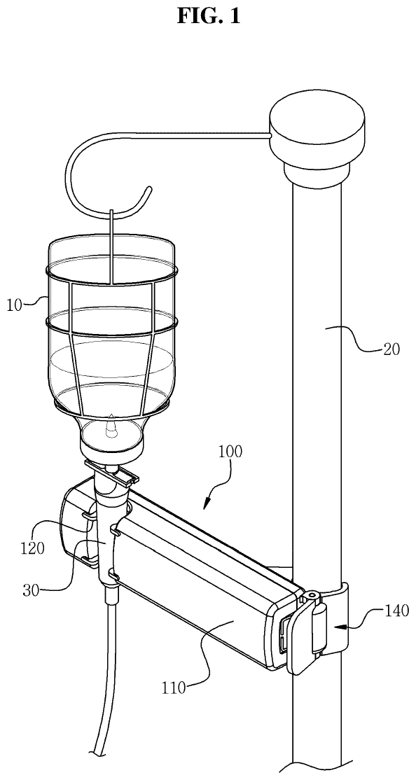

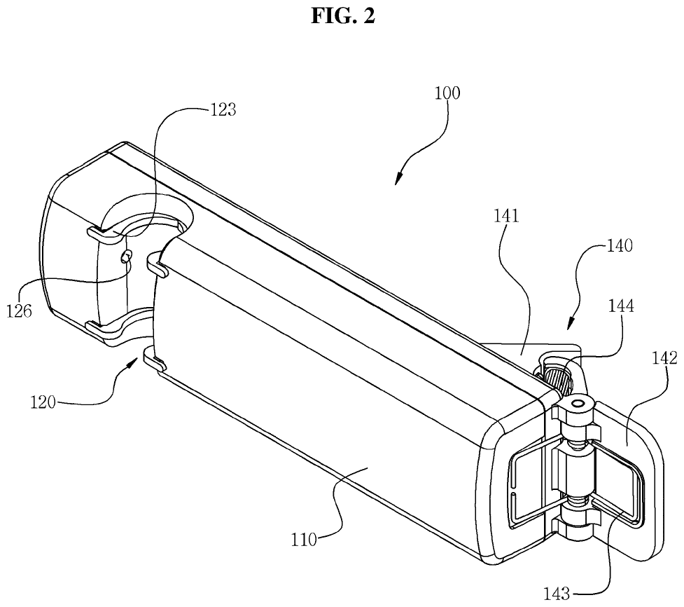

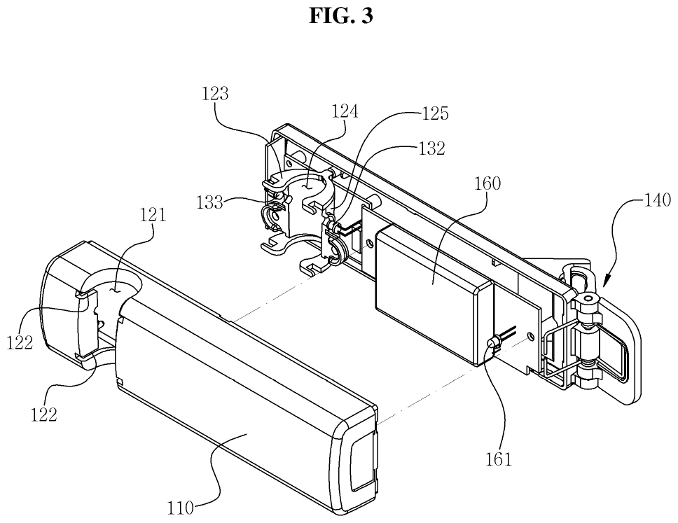

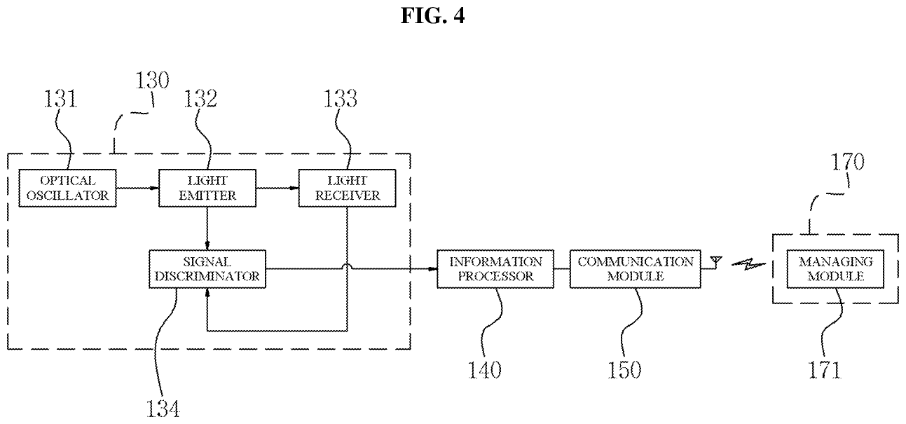

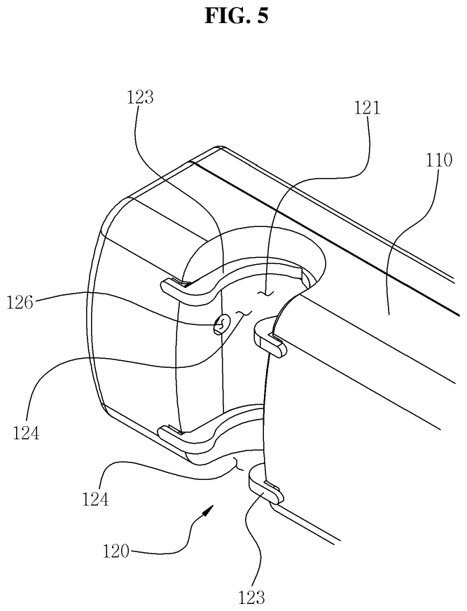

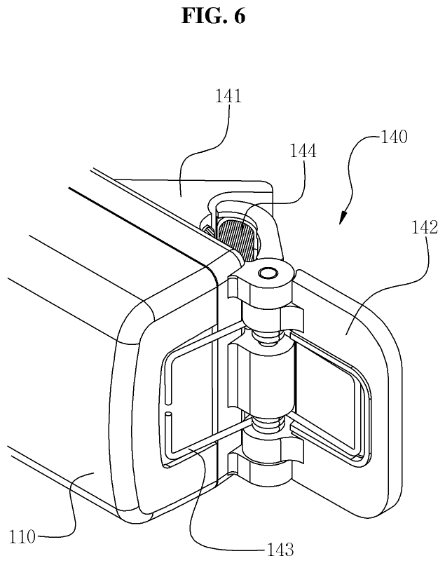

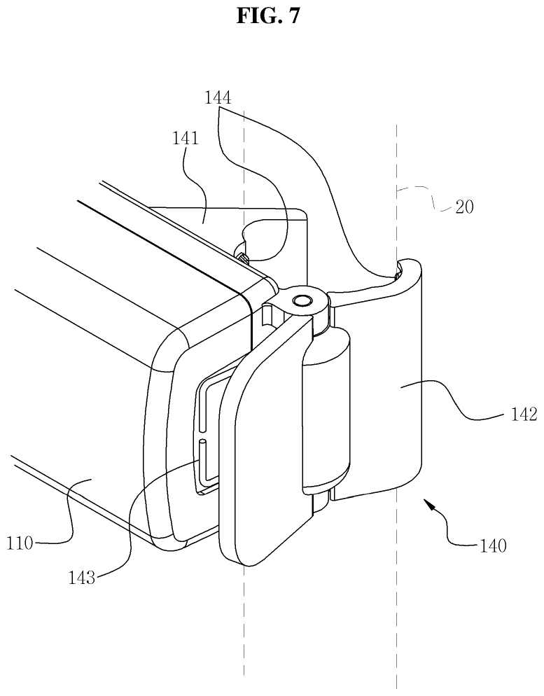

### DETAILED DESCRIPTION

Hereinafter, a Ringer's solution monitoring apparatus according to an embodiment of the present invention will be described with reference to the accompanying drawings. Since the present invention allows for various changes and numerous embodiments, specific embodiments will be illustrated in the drawings and described in detail in the written description. However, this is not intended to limit the present invention to specific embodiments, and it is to be appreciated that all changes, equivalents, and substitutes that do not depart from the spirit and technical scope of the present invention are encompassed in the present invention. Like numbers refer to like elements throughout the description of the figures. In the accompanying drawings, sizes of structures may be greater than those of actual structures for clarity of the present invention or may be smaller than those of the actual structure such that a schematic structure of the present invention is understood.

It will be understood that, although the terms “first,” “second,” etc. may be used herein to describe various elements, these elements should not be limited by these terms. These terms are only used to distinguish one element from another. For example, a first element could be termed a second element, and a second element could similarly be termed a first element without departing from the scope of the present invention.

The terminology used herein is for the purpose of describing particular embodiments only and is not intended to be limiting to the invention. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements, components, and/or groups thereof but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined here.

Referring to the drawings, a Ringer's solution monitoring apparatus 100 according to the present invention includes a main body 110 installed on a hanger 20 on which a Ringer's solution container 10 is hung, a dropper attachment part 120 which is provided in the main body 110 and to which a dropper 30 is attached, a Ringer's solution detector 130 which detects a state in which a Ringer's solution falls, and a fixing unit 140 configured to fix the main body 110 to the hanger 20.

Since the fixing unit 140 is provided one side of the main body 110, the main body 110 may be attached to the hanger 20.

The fixing unit 140 includes a fixing support 141 formed one side of a rear surface of the main body 110, a rotating support 142 configured to rotate toward the fixing support 141 to grasp the hanger 20, and an elastic member 143 which is elastically biased such that the rotating support 142 rotates toward the fixing support 141.

The fixing support 141 is formed to be in contact with one side of an external surface of the hanger 20, and the rotating support 142 is formed such that an inner circumferential surface thereof wraps around the external surface of the hanger 20 and grasps the hanger 20 with the fixing support 141.

Particularly, since friction pads 144 are provided on inner contact surfaces, which are in contact with the hanger 20, of the fixing support 141 and the rotating support 142, the fixing unit 140 does not slide downward along a surface of the hanger 20 in a state in which the friction pads 144 are in contact with the hanger 20, and thus the fixed state can be easily maintained. The friction pads 144 may be formed of rubber or a synthetic resin on which a surface treatment is performed to increase a coefficient of friction.

In addition, the elastic member 143 is a torsion spring in which one end thereof is supported by a side surface portion of the main body 110 and the other end is supported by the rotating support 142, and a state in which the rotating support 142 has rotated toward the fixing support 141 may be maintained by the elastic member 143.

Since an outer diameter or a material of the hanger 20 is different in each hospital, when the rotating support 142 is opened to position the hanger 20 between the rotating support 142 and the fixing support 141 and then the rotating support 142 is released, the rotating support 142 grasps the hanger 20 due to the elastic member 143 and maintains a fixed state of the main body 110. In addition, when the main body 110 is removed from the hanger 20 for charging or another reason, the rotating support 142 may be opened to simply remove the main body 110 from the hanger 20.

The dropper attachment part 120 is to fix the dropper 30 at a position at which the Ringer's solution detector 130 may easily detect a falling state of a Ringer's solution.

To this end, the dropper attachment part 120 is provided at one side of a front surface of the main body 110 and includes an inlet groove 121 recessed a predetermined depth from the front surface of the main body 110 in a rearward direction and dropper pressing parts 123 configured to fix the dropper 30 which has entered the inlet groove 121.

The inlet groove 121 may be formed to have a diameter which is greater than an external diameter of the dropper 30, and long holes 122 are formed at upper and lower portions of the inlet groove 121 to extend in a lateral direction so as to install the dropper pressing parts 123 which will be described below.

In addition, an optical sensor mounting hole 126 is formed between the long holes 122 in the inlet groove 121 such that optical sensors of the Ringer's solution detector 130, which will be described below, may emit light toward the dropper 30 or may receive light.

Inlet openings 124 are formed in the dropper pressing parts 123 such that the dropper 30 enters in a direction corresponding to an opening of the inlet groove 121, and the inlet openings 124 are fixed to a supporting rod 125 which vertically extends in the main body 110.

In addition, the both end portions of the dropper pressing parts 123 with respect to the inlet opening 124 may be elastically deformed to be opened laterally along an external diameter of the dropper 30.

Accordingly, when the dropper 30 is inserted into the inlet groove 121 and installed in the dropper attachment parts 120, the dropper 30 enters inner sides of the dropper pressing parts 123 through the inlet opening 124, and the both end portions of the dropper pressing parts 123 are opened so that the dropper 30 enters the inlet groove 121. After the dropper 30 enters the inlet groove 121, a fixed state of the dropper 30 is maintained due to elastic forces by which an initial state of the both end portions of the dropper pressing parts 123 is restored, and when an external force is applied to remove the dropper 30, the end portions of the dropper pressing parts 123 are opened so that the dropper 30 is removed through the inlet opening 124.

Since the dropper pressing parts 123 are capable of being elastically deformed such that the end portions thereof are opened, the dropper 30 having various sizes can be installed.

In the case of the present embodiment, even though two dropper pressing parts 123 are formed to be vertically spaced apart from each other, the dropper pressing part 123 may be formed at one side of the upper and lower portions of the inlet groove 121, and three or more dropper pressing parts 123 may also be disposed to be vertically spaced apart from each other.

The Ringer's solution detector 130 includes an optical oscillator 131 configured to oscillate light, a light emitter 132, a light receiver 133, and a signal discriminator 134. The Ringer's solution detector 130 may detect light such as infrared light, laser, and ultraviolet light in various wavelengths, and light oscillated by the optical oscillator 131 is emitted from the light emitter 132 toward the light receiver 133. The light receiver 133 receives the light emitted from the light emitter 132, and at this time, in a case in which the light passes between the light emitter 132 and the light receiver 133, the light emitted from the light emitter 132 is not received by the light receiver 133 or is deformed due to the Ringer's solution. The light receiver 133 transmits a light receiving signal to the signal discriminator 134, and the signal discriminator 134 determines whether the Ringer's solution is falling using the light receiving signal of the light receiver 133.

Here, the light emitter 132 and the light receiver 133 are installed on an internal surface of an insertion groove 123 and may be installed on supporting pads 122 positioned at positions facing each other with respect to a vertical central line of the insertion groove 123.

Here, although the signal discriminator 134 is not illustrated in the drawings, the signal discriminator 134 is installed in a case 111, also collects a light emitting signal when the light emitter 132 emits light, and discriminates the signal. This is to prevent a detection error due to natural light. In a case in which natural light is incident in the insertion groove 123, since a detection error may occur due to the natural light when light is received by the light receiver 133 even though the Ringer's solution passes between the light emitter 132 and the light receiver 133 and falls, the signal discriminator 134 determines that a signal of a light receiving signal of the light receiver 133 is a normal signal when the light emitting signal of the light emitter 132 is the same as the light receiving signal of the light receiver 133 and determines that the Ringer's solution is falling only when the light receiving signal is not received in a state in which the light emitting signal is received.

Information discriminated by the signal discriminator 134 is transmitted to an information processor 140, and information of an input amount, an input speed, and the like of the Ringer's solution is calculated. However, the information processor 140 may be provided in the main body 110, and the information processor 140 may also be formed such that information discriminated by the signal discriminator 134 is transmitted to a wireless terminal 170 of a user through a communication module 150 which will be described below, the information is processed by the wireless terminal 170, and information of an input speed or an input amount may be obtained.

The communication module 150 configured to transmit detected information detected by the Ringer's solution detector 130 may be provided in the main body 110, a manager configured to obtain and manage Ringer's solution detection information may include the wireless terminal 170 capable of receiving and transmitting information, and the wireless terminal 170 may include a managing module 171 configured to receive and display the detected information transmitted through the communication module 150.

The communication module 150 may use various wireless communication methods such as a Bluetooth beacon or Wi-Fi technology.

In the case in which the Wi-Fi technology is applied, since access points (APs) for Wi-Fi communication are installed in each patient's room in which patients are positioned or in predetermined sections in a hospital, the communication module 150 transmits the detected information through the APs, the transmitted information is transmitted to terminals 170 of nurses who are managers through a wireless internet communication network or a wired communication network. Here, the terminal 170 may be a portable wireless terminal such as a smartphone or a tablet personal computer (PC), a notebook, or a desktop PC.

The communication module is configured such that all AP lists present in the hospital are stored therein, and in a case in which a user goes to a toilet or moves to another location from the patient's room for other examination, treatment, or diagnosis, although the communication module is disconnected from a presently connected AP, the communication module is connected to an AP capable of being immediately connected thereto among APs in the stored AP lists, and the detected information may be continuously transmitted thereto.

In addition, a battery 160 supplying power for driving the Ringer's solution detector 130 or the communication module 150 is provided in the main body 110.

Although a separate charging cable may also be connected to the battery 160 so as to charge the battery 160, a charging terminal (not shown) may be provided at one side of a side surface of the main body 110, a charging cradle (not shown) into which the main body 110 is inserted may be provided, and charging of the main body 110 may be performed. In this case, a coupling terminal in contact with the charging terminal may be provided in the charging cradle such that a power source is connected to the charging cradle.

In addition, a component 161, which is not described, is a display lamp 161 that displays a charging state and a device operating state of the battery 160. The component 161 displays an operating state or a shortage state of the battery 160 using a color of a lamp or a flashing state of the lamp.

As described above, since a Ringer's solution monitoring apparatus can be installed on Ringer's solution hangers having various sizes, there is an advantage in that ease of use is improved, and since a manager can remotely check information about an injection speed and an injection amount of a Ringer's solution in real time, there is an advantage in that efficiency of injection management for the Ringer's solution is improved.

The description about the disclosed embodiments is provided for those skilled in the art to use or implement the present invention. Various modifications of the embodiments will be clear to those skilled in the art, and general principles defined in the present specification may be applied to other embodiments without departing from the scope of the present invention. Therefore, the present invention is not limited to the embodiments disclosed in the present specification and should be interpreted within the widest scope consistent with the principles and novel features disclosed in the present specification.

What is claimed is:

1. A Ringer's solution monitoring apparatus comprising:
a main body;
a dropper attachment part which is provided on the main body and to which a dropper is attached;
a Ringer's solution detector configured to detect a Ringer's solution falling in the dropper attached to the dropper attachment part; and
a fixing unit configured to detachably fix the main body to a hanger on which a Ringer's solution container is hung.

2. The Ringer's solution monitoring apparatus of claim 1, wherein:
the main body has a predetermined inner space therein; and
the Ringer's solution detector is attached into the main body.

3. The Ringer's solution monitoring apparatus of claim 2, wherein:
the dropper attachment part includes an inlet groove formed to be recessed a predetermined depth in an inward direction such that the dropper is inserted into one side of an external surface of the main body;
the Ringer's solution detector includes an optical sensor configured to determine whether the Ringer's solution is falling in the dropper using light; and
a light emitter and a light receiver are formed in a surface of the inlet groove to emit light toward the dropper which has entered the inlet groove and to receive light.

4. The Ringer's solution monitoring apparatus of claim 3, wherein:
the dropper attachment part further includes a dropper pressing part configured to press a surface of the dropper which has entered the inlet groove and to maintain a state in which the dropper has entered the inlet groove; and
the dropper pressing part includes an inlet opening formed in a direction corresponding to an opening of the inlet groove such that the dropper enters and is formed such that both ends of the dropper pressing part are opened outwardly along an external diameter of the dropper and are elastically deformable.

5. The Ringer's solution monitoring apparatus of claim 4, wherein the dropper pressing part includes a plurality of dropper pressing parts formed to be vertically spaced apart from each other in the inlet groove.

6. The Ringer's solution monitoring apparatus of claim 1, wherein the fixing unit includes:
a fixing support which is formed at one side of a rear surface of the main body and wraps around one side of an outer circumferential surface of the hanger;
a rotating support which is formed to be rotatable on the rear surface of the main body and wraps the outer circumferential surface of the hanger with the fixing support; and
an elastic member which elastically biases the rotating support in a direction in which the hanger is pressed such that a state in which the main body is fixed to the hanger is maintained by maintaining a state in which the fixing support and the rotating support press the outer circumferential surface of the hanger.

7. The Ringer's solution monitoring apparatus of claim 6, wherein:
the fixing support and the rotating support are allowed to wrap around the hanger regardless of an external diameter of the hanger because the rotating support rotates; and
a friction pad is formed on a contact portion in contact with the outer circumferential surface of the hanger so as to increase a frictional force.

8. The Ringer's solution monitoring apparatus of claim 1, further comprising:
a communication module which transmits detected information detected by the Ringer's solution detector; and
a managing module which is installed in a terminal of a user and receives and displays the detected information transmitted from the communication module through the terminal of the user.

9. The Ringer's solution monitoring apparatus claim 8, wherein any one wireless communication method among Bluetooth, Bluetooth beacon, and Wi-Fi is applied to the communication module.

10. The Ringer's solution monitoring apparatus of claim 1, wherein:
a battery configured to supply power for driving the Ringer's solution detector is installed in the main body;
a charging terminal is provided on a side surface of the main body to charge the battery; and
a cradle in which a coupling terminal in contact with the charging terminal of the main body is provided and the main body is inserted thereinto to charge the battery is further included.

11. The Ringer's solution monitoring apparatus claim 8, wherein:
a Wi-Fi wireless communication method is applied to the communication module; and
the communication module is formed such that an access point (AP) list within a moving range in which the main body moves is stored in the communication module, and even when the main body moves and is disconnected from a currently connected AP, the main body is connected to a new AP in the stored AP list.
